(19) United States
(12) Patent Application Publication
(10) Pub. No.: US 2012/0065497 A1
(43) Pub. Date: Mar. 15, 2012

(54) Three Dimensional Minimally-Invasive Spinal Imaging System and Method

(75) Inventors: Richard L. Brown, Mesa, AZ (US); Erik J. Herrmann, Mesa, AZ (US)

(73) Assignee: Warsaw Orthopedic, Inc., Warsaw, IN (US)

(21) Appl. No.: 12/879,752

(22) Filed: Sep. 10, 2010

(51) Int. Cl. A61B 8/08

(52) U.S. Cl. 600/424

(57) ABSTRACT

A method and system is disclosed that is operable to generate a location value associated with an implant that has been implanted in a predetermined location of a vertebra of a spine. The location value can be utilized to generate a three-dimensional animation of the spine in motion. The system includes a plurality of implants that include a controller having a memory. The implants also include a telemetry unit connected with the controller that is used to wirelessly transmit and receive data. In addition, the implants include an acoustic generator that is configured to generate an acoustic pulse in response to a signal from the controller. An external control unit is wirelessly connected with the implant for receiving various data values from the implant.

### BACKGROUND

The present invention relates generally to imaging apparatus and systems and more particularly, to a system and method for minimally-invasively generating three dimensional images of a human spine during movement of the spine using acoustic signals.

Plain radiographs of the spine are useful in evaluating the vertebral column for position of bones, continuity of the spinal column and spinal canal, bone density, changes in the articular facet joints, and integrity of vertebral body end plates and disc spaces. Good positioning and radiographic techniques are critical for evaluating the spine. To monitor the relative location of the vertebra of the spine during movement, multiple radiographs must be taken which exposes the patient to various amounts of radiation. Myelography involves the injection of iodinated contrast into the subarachnoid space. Radiographs performed following this injection allow visualization of the outline of the spinal cord. Compressive lesions can be readily seen using this technique. Disc extrusion or extradural spinal cord tumors are commonly diagnosed with a myelogram.

CT (computed tomography) is basically radiography performed in an axial plane. CT is excellent for evaluation of bone detail, and is ideal for visualizing bone tumors, spinal fractures, and discospondylitis. Soft tissues are better visualized using CT than on plain radiographs, but interpretation of subtle soft tissue lesions can still be difficult. MRIs allow the best visualization of soft tissue lesions, and is especially useful for diagnosing intramedullary spinal cord lesions, such as neoplasms, syringomyelia, arachnoid cysts, and even occasionally infarcts. Although MRI technology is minimally-invasive, it still does require significant time to perform. In addition, it is the most expensive imaging modality and because of the time required, scanning of a large area (such as the thoracolumbar spine) can be problematic. Further, the use of an MRI is extremely expensive and may not always be available in rural areas or developing countries.

For some applications, it would be beneficial to monitor the movement of the spine utilizing a minimally invasive means that is inexpensive and can be performed by almost any physician and at much less expense that existing technology. As such, a need exists for a minimally invasive system that will allow physicians to monitor the movement of the vertebra of the spine when diagnosing problems of the spine.

### SUMMARY

According to one aspect a system is disclosed that is configured to generate a location value associated with an implant that has been implanted in a predetermined location of a vertebra of a spine. The location value can be utilized to generate a three-dimensional animation of the spine in motion. The system includes a plurality of implants placed in the predetermined location on a plurality of respective vertebra of a patient. The implants include a controller having a memory that is used to store various data values as well as, in some forms, software applications used to control the various circuitry of the implant. The implants also include a telemetry unit connected with the controller that is used to wirelessly transmit and receive data. In addition, the implants include an acoustic generator that is configured to generate an acoustic pulse in response to a signal from the controller.

An external control unit is in communication with the telemetry unit of the implant. The external control unit is operable to generate a reading signal that is sent to the implant via the telemetry unit that causes the implant to generate the signal. At least three external receiving patches are attached to an exterior surface of the patient and connected with the external control unit. The external control unit is operable to generate a first time stamp associated with the reading signal and a plurality of unique time stamps associated with times at which the at least three external receiving patches detect the acoustic pulse.

The external control unit is operable to generate a three-dimensional location reading as a function of the first time stamp and the plurality of unique time stamps. The first time stamp is associated with a time in which the signal is generated. In some forms, a gyroscope sensor is connected with the controller that is configured and operable to generate an angular rotation reading associated with each of the implants. The angular rotation reading is transmitted to the control unit using the telemetry unit. In other forms, an accelerometer is connected with the controller that is configure and operable to generate an angular motion reading associated with each of the implants. The angular motion reading is transmitted to the control unit using the telemetry unit as well. The angular motion reading corresponds to and is indicative of lateral bending and flexion extension of each of the vertebra to which an implant is connected. A spinal animation application is included that is configure to generate a spinal animation as a function of a plurality of readings taken from the implants.

According to another aspect a system is disclosed that is configured to generate a location value associated with an implant that has been implanted in a predetermined location of a vertebra of a spine. The location value can be utilized to generate a three-dimensional animation of the spine in motion. The system includes at least one implant configured to be implanted on a vertebra of a spine. The implant includes a controller connected with an acoustic generator that is operable to generate an acoustic pulse in response to a signal from the controller. The implant also includes a gyroscope connected with the controller configured and operable to generate an angular rotation reading associated with the vertebra. Further, the implant includes an accelerometer connected with the controller that is configured and operable to generate an angular motion reading associated with the vertebra. A first telemetry unit is connected with the controller that is configured to transmit and receive wireless signals.

The system also includes an external control unit that is connected with a second telemetry unit in communication with the first telemetry unit of the implant. The external control unit is also connected with at least three external receiver patches that are placed on a patient's skin in a spaced apart relationship in relation to the at least one implant. The external control unit is operable to generate a reading signal that is transmitted to the controller of the implant as well as associate a first time value with the reading signal.

In response to the reading signal, the controller is operable to generate the signal to cause the acoustic generator to generate the acoustic pulse. In response to the reading signal the controller obtains and transmits said angular rotation reading and said angular motion reading to said external control unit, wherein each of said external receiving patches are operable to generate detection signals that are transmitted to said external control unit when said acoustic pulse is detected by said external receiving patches, and wherein said external control unit is operable to assign a detection time value to each detection signal received from each respective external receiving patch.

In one representative form, the external control unit includes a universal interface port for transmitting data to a computing device. The system can also include an implant location application operable to determine a location of the implant as a function of the first time value and the detection time values. During operation, a plurality of reading signals are generated to generate a plurality of result sets that are stored in a database. Each result set includes the first time value, the detection time values, the angular rotation reading and the angular motion reading. The system can further include an animation application configured to generate a spinal animation as a function of the plurality of result sets.

In yet another representative form, the system includes a temperature sensor that is connected with the controller for generating a temperature reading of tissue surrounding the at least one implant. In another form, a rechargeable power unit is connected with the controller, the gyroscope sensor, the accelerometer, and the acoustic generator. The system can include an external power patch that is placed on the patient's skin for recharging the rechargeable power unit. In one form, the power patch is operable to generate electromagnetic energy that is used to charge the rechargeable power unit. In yet another form, the power patch is operable to generate acoustic waves that are used to charge the rechargeable power unit.

Yet a further aspect discloses a method of generating a location value associated with an implant that has been implanted in a predetermined location of a vertebra of a spine. The location value can be utilized to generate a three-dimensional animation of the spine in motion. The method includes the steps of: generating a reading request with an external control unit that is wirelessly transmitted to at least one implant oriented in a predetermined location on a vertebra of a spine; recording a first time value associated with the reading request; generating an acoustic ping with the implant in response to the reading request; obtaining an angular rotation reading and a angular movement reading in response to the reading request; wirelessly transmitting the angular rotation reading and the angular movement reading to the external control unit; monitoring at least three external receiving patches connected with the external control unit for detection of the acoustic ping; recording a detection time value from each the external receiving patch when the acoustic ping is detected; storing the first time value, the angular rotation reading, the angular movement reading, and the detection time values in a database associated with the external control unit; calculating a location value for the at least one implant as a function of the first time value and the detection time values; and generating a graphical animation of the implants on the vertebra of the spine as a function of the location value, the angular rotation reading, and the angular movement reading.

Related features, aspects, embodiments, objects and advantages of the present invention will be apparent from the following description.

### BRIEF DESCRIPTION OF THE DRAWINGS

FIG. 1 illustrates a minimally invasive spinal imaging system that uses acoustic pulses to generate spinal images.

FIG. 2 is the minimally invasive spinal imaging system shown in FIG. 1 illustrating an acoustic pulse.

FIG. 3 is a representative implant of the spinal imaging system illustrated in FIG. 1.

FIG. 4 is a block diagram of the electronic circuitry contained in the implant.

FIG. 5 is a block diagram of the electronic circuitry contained in an external control unit of the spinal imaging system.

FIG. 6 is a flow chart illustrating representative operational steps of the spinal imaging system.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended. Any such alterations and further modifications in the illustrated devices, and such further applications of the principles of the invention as illustrated herein are contemplated as would normally occur to one skilled in the art to which the invention relates.

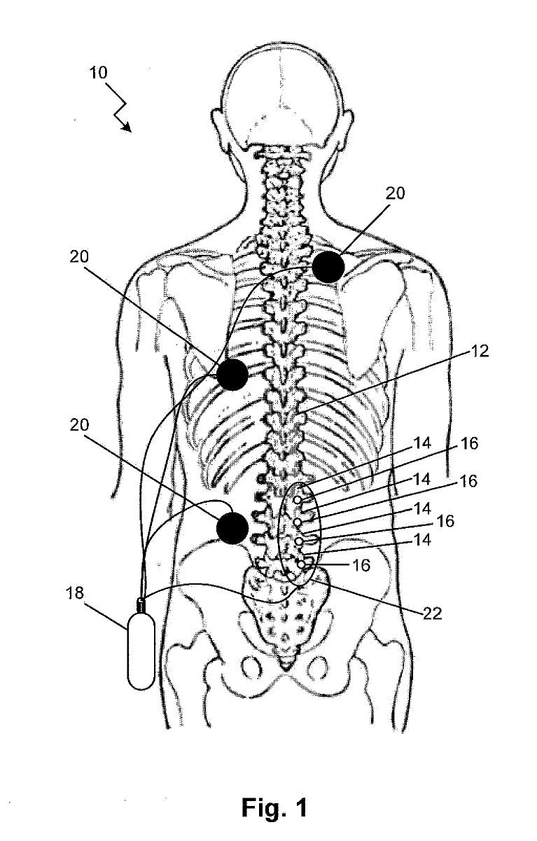

Referring to FIG. 1, shown therein is a spinal motion tracking system 10 that is capable of generating a three dimensional graphical representation of a portion of a spine 12. The three dimensional graphical representation can be generated in a stationary position, in which a patient is holding still, or in a moving position where the patient is asked to move in certain directions in order to generate an animated three dimensional graphical representation of the portion of the spine 12 so that a medical provider can view the movement of various vertebra 14 of the spine 12. The spinal motion tracking system 10 does not use any radiation to track the movement of the vertebra 14 of the spine 12 and can be utilized in a minimally-invasive manner that does not require hospitalization. The procedure to implant and measure the motion is simple enough that primary physicians or physicians without access to expensive imaging equipment can utilize the diagnosing and monitoring equipment.

As illustrated in FIG. 1, the system 10 includes one or more compression wave signal emitting implants 16. In one form, the signal emitting devices 16 are operable to generate acoustic signals. In the representative form, at least one signal emitting implant 16 is implanted on one or more vertebra 12 at a predetermined location of the vertebra 12. In the illustrated form, the signal emitting implants 16 are screwed into the vertebra 14 but other attachment mechanisms may be used as well. In some forms, a plurality of signal emitting implants 16 can be implanted on an individual vertebra 12 or multiple vertebrae 12 for a more detailed analysis of the movement of the respective vertebra 12 to be analyzed. The signal emitting implants 16 have been implanted on the lamina of the vertebra 12 in this form, but it should be appreciated that the signal emitting implants 16 could be inserted on other locations as well such as the spinous process, transverse process, superior and inferior articular process, pedicals, and vertebral body. In other forms, the implants 16 can be installed in any semi-rigid structure in which movement is desired to be monitored within the human body.

The system 10 also includes an external control unit 18 that is connected with at least three external compression wave detection or receiver patches 20. The external receiver patches 20 are adhesive patches that include a receiver operable to detect emitted pulses. In the illustrated form, the control unit 18 comprises a hand held or belt worn controller. In other forms, the control unit 18 can comprise a bench-top computing system having a display. The control unit 18 is operable to cause the signal emitting implants 16 to generate compression waves or acoustic signals that are transmitted through the body tissue. The detection patches 20 receive these signals and as set forth below, the system 10 processes these signals to provide a point in three-dimensional space. In one form, the control unit 18 is also connected with a power patch 22. The power patch 22 can be used to generate electromagnetic energy that is used to charge the signal emitting implants 16. In another form, the power patch 22 can be used to generate acoustic waves that charge the signal emitting implants 16.

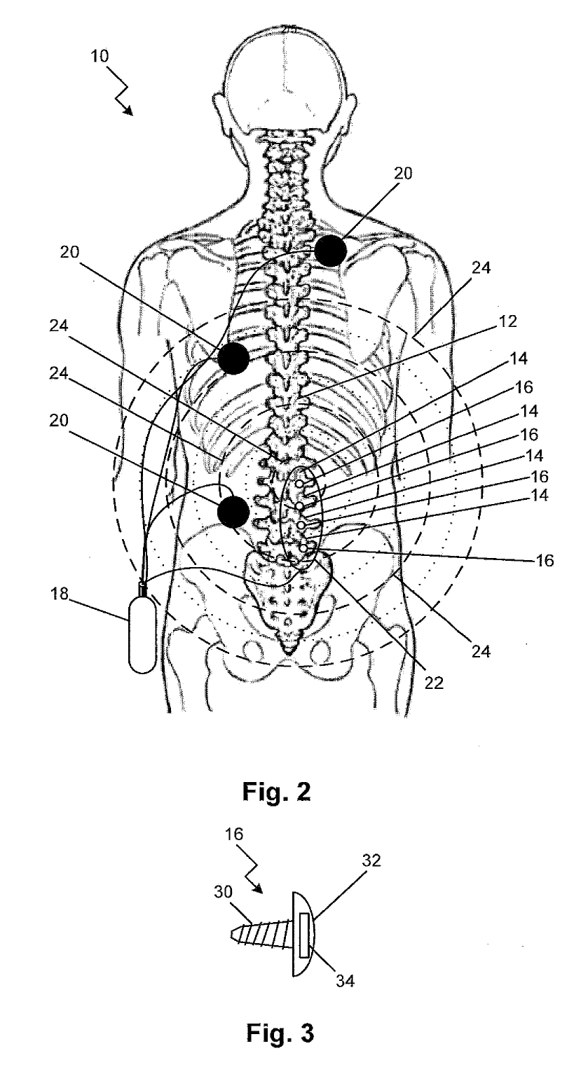

Referring to FIG. 2, in response to a wireless location signal or instruction generated and transmitted by the control unit 18, the upper most signal emitting implant 16 is illustrated generating an acoustic pulse 24. The acoustic pulse 24 generates a wavefront that radiates outwardly from each respective signal emitting implant 16 toward each respective detection patch 20. The detection patches 20 are operable to detect these pulses and generate a detection signal that is sent to the control unit 18. If the control unit 18 comprises a computing system, a three-dimensional representation or animation of the movement of the spine 12 is generated on a display that illustrates how the spine 12 actually looks as the patient is moving.

One or all of the signal emitting implants 16 can generate an acoustic pulse in response to the location signal transmitted by the control unit 18 to the signal emitting implants 16. The signal emitting implants 16 can be stimulated in sequential order or a predetermined order. The signal emitting implants 16 could also be stimulated simultaneously if each signal emitting implant 16 is programmed or designed to emit acoustic pulses 24 using different predetermined frequencies. In some forms, a transmitter 22 is connected with the control unit 18 and placed in close proximity to the signal emitting implants 16. The transmitter 22 is responsible for sending operational instructions to the signal emitting implants 16.

Referring to FIG. 3, a representative signal emitting implant 16 is illustrated. In this form, the signal emitting implant 16 comprises a screw. The signal emitting implant 16 includes a threaded shaft 30 connected with a head portion 32. In one form, the head portion 32 of the signal emitting device 16 includes an electronic circuit 36 that is used to generate the acoustic pulse 24. In other forms, a portion of the electronic circuit 36 could be located in the threaded shaft 30 as well. The signal emitting implant 16 is a miniature device that can be installed using a minimally invasive procedure. In one form, the head portion 32 of the screw has a diameter of about 10 millimeters and a width of about 3 millimeters. The threaded shaft 30 can have a length of about 8 millimeters.

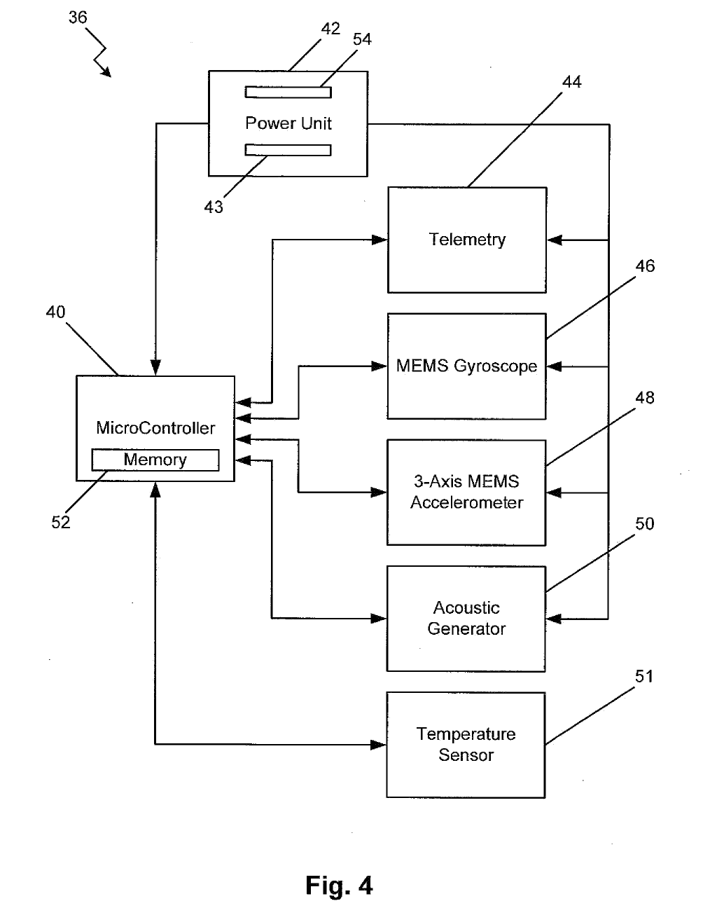

Referring to FIG. 4, the electronic circuit 36 of the signal emitting implant 16 that is implanted into the vertebra 14 of the spine 12 of the patient is set forth in detail. The electronic circuit 36 includes a micro-controller 40, a power unit 42, a telemetry or transceiver unit 44, a microchip-packaged MEMS gyroscope sensor 46, a three-axis MEMS accelerometer 48, and an acoustic generator 50. In one form, the controller 40 also includes a memory unit 52 that is used to store software designed to operate the electronic circuit 36 as well as readings taken from the sensors for transmission to the control unit 18. In a representative form, the controller 40 comprises a microprocessor based controller, an application specific integrated circuit (ASIC), or a combination of both digital and analog circuitry. The controller 40 can also be connected with a temperature sensor 51. The temperature sensor 51 is operable to generate a temperature reading that is transmitted to the controller 40, which in turn can transmit the temperature reading to the external control unit 18 via the telemetry unit 44. Monitoring the temperature of the tissue surrounding the area that the implant 16 is implanted can be utilized to detect infection in the tissue surrounding the area where the implant 16 has been implanted.

The power unit 42 is connected with and used to provide power to each of the above-referenced components in the electronic circuit 36. In one form, the power unit 42 comprises one or more batteries 43 for a single use or one or more rechargeable batteries 43 for multiple uses. In addition to one or more batteries, the power unit 42 can also include a charge circuit 54 that is connected with the batteries 43. The charge circuit 54 is used to charge the batteries 43 in the forms in which the batteries 43 comprise rechargeable batteries. Referring to FIGS. 1 and 4, the control unit 18 can be connected with a power patch 22 that is operable to generate electromagnetic waves that cause the charge circuit 54 to charge the batteries 43. In yet another form, the control unit 18 can be connected with an acoustic waveform generator 22 that excites the acoustic generator 50 of the electronic circuit 36 thereby charging the batteries 43. In this form, the acoustic generator 50 is operated one way to provide current to the charge circuit 54 and then reversed when not charging to emit the acoustic signal or pulse 24.

As illustrated in FIG. 4, the controller 40 is connected with the telemetry unit 44. The telemetry unit 44 is operable to wirelessly send and receive signals to and from the control unit 18. The controller 40 is also connected with the gyroscope 46, which is operable to measure the angular rotation of the vertebra 14. In operation, the gyroscope 46 generates an angular rotation reading that is sent to the controller 40. In one form, the angular rotation reading is stored in the memory 52 of the controller 40 and then transmitted to the control unit 18. In another representative form, the controller 40 can transmit the angular rotation reading to the control unit 18 using the telemetry unit 44 after the acoustic pulse 24 is generated. After one or more readings are obtained from each signal emitting implant 16, the readings are used to generate a representation of the angular movement of the vertebra 14 while the patient moves.

The controller 40 is also connected with the accelerometer 48. The accelerometer 48 is operable to generate an angular motion reading of the vertebra 14. The angular motion reading is indicative of the lateral bending and flexion extension of the vertebra 14 of the spine 12. The angular motion reading is transmitted to the controller 40, which in one form stores the angular motion reading in the memory 52. In other forms, the angular motion reading can be transmitted to the control unit 18 along with other readings using the telemetry unit 44.

The controller 40 is also connected with the acoustic generator 50. The acoustic generator 48 can comprise a MEMS speaker, a piezoelectric transducer, or any other miniature device capable of generating an acoustic pulse 24. As previously set forth, the signal emitting implant 16 is utilized to generate a waveform via the acoustic generator 50, which can be referred to as a “ping”, that is sensed by the detection patches 20. The controller 40 causes the acoustic generator 48 to generate the acoustic pulse 24 in response to a signal received from the control unit 18. In particular, the controller 40 senses a communication signal received from the control unit 18 via the telemetry unit 44. In response to this signal, the controller 40 instructs the acoustic generator 50 to generate the acoustic pulse 24. At the same time, the controller 40 records the time that the acoustic generator 50 generates the acoustic pulse and stores a time stamp associated with the acoustic pulse in memory 52. In alternative forms, the external control unit 18 can record the time in which an instruction is sent to the controller 40 of the implant 16 to cause the acoustic generator 50 to generate a ping.

Referring to FIGS. 2 and 4, as the acoustic pulse 24 travels through the patient's body it will eventually reach each respective detection patch 20. The detection patches 20 are located on various portions of the patient's body in a spaced apart relationship such that the acoustic pulse reaches each detection patch 20 at a different time. In some forms, the controller 40 then automatically takes a reading from the gyroscope 46 and the accelerometer 48 and transmits the readings to the control unit 18 using the telemetry unit 44. Thus, the control unit 18 is configured to store a time stamp associated with the time each ping is generated by the acoustic generator 50, a set of time stamps associated with the times in which each ping is detected by each respective detection patch 20, an angular rotation reading detected by the gyroscope 46, and an angular motion reading detected by the accelerometer 48.

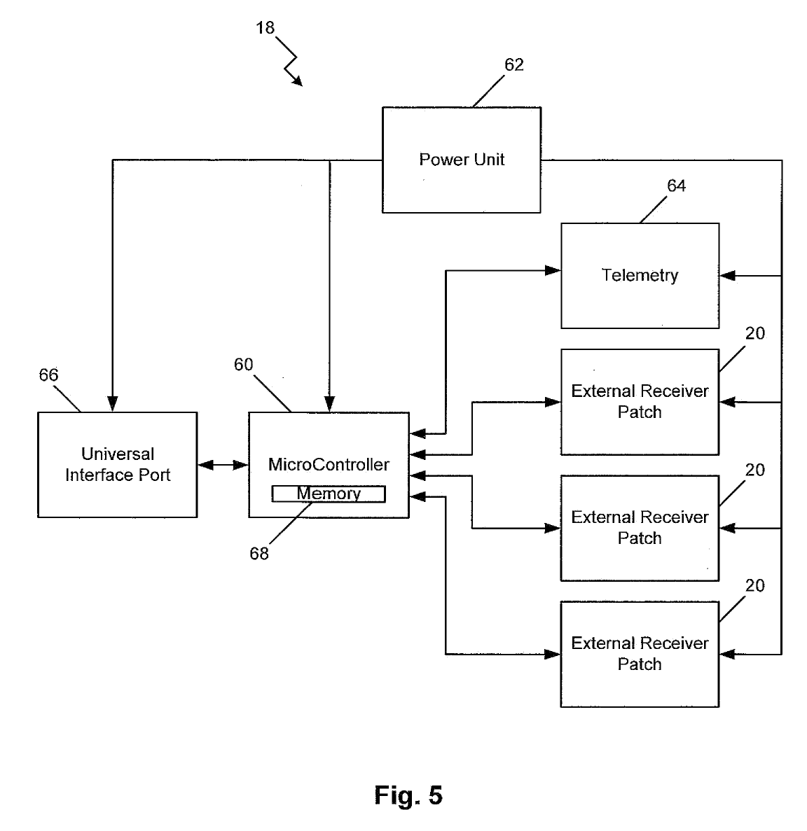

Referring to FIGS. 2 and 5, a representative control unit 18 is illustrated in the form of a handheld device. In this form, the handheld device 18 includes a micro-controller 60, a power unit or supply 62, a telemetry unit or transceiver 64, and a universal interface port 66. The controller 60 is connected with each one of the respective detection patches 20. If necessary, the detection patches 20, the micro-controller 60, the telemetry unit 64, and the universal interface port 66 can be connected with the power unit 62. The power unit 62 can comprise a standard power supply, one or more batteries, or one or more rechargeable batteries. The micro-controller 60 includes a memory device 68 that is used to store software and data received from readings received via the telemetry unit 64. In other forms, the controller 60 can comprise a micro-processor, a digital signal processor, or a combination of digital and analog circuitry. In this form, the universal interface port 66 is configured to be connected with a personal computer or workstation to transmit or download the data readings to a 3-D animation software application used to display a graphical representation of the motion of the spine 12.

The telemetry unit 64 of the control unit 18 can be an integral part of the control unit 18. In other forms, the telemetry unit 64 could be a separate unit connected with the control unit 18. As such, the telemetry unit 64 may be connected with the patient or placed in close proximity to the implants 16 in the patient. The manner in which the telemetry unit 64 communicates with the implants 16 could be accomplished via electromagnetic communication, radio frequency identification, or conductive communication using the body as a signal path.

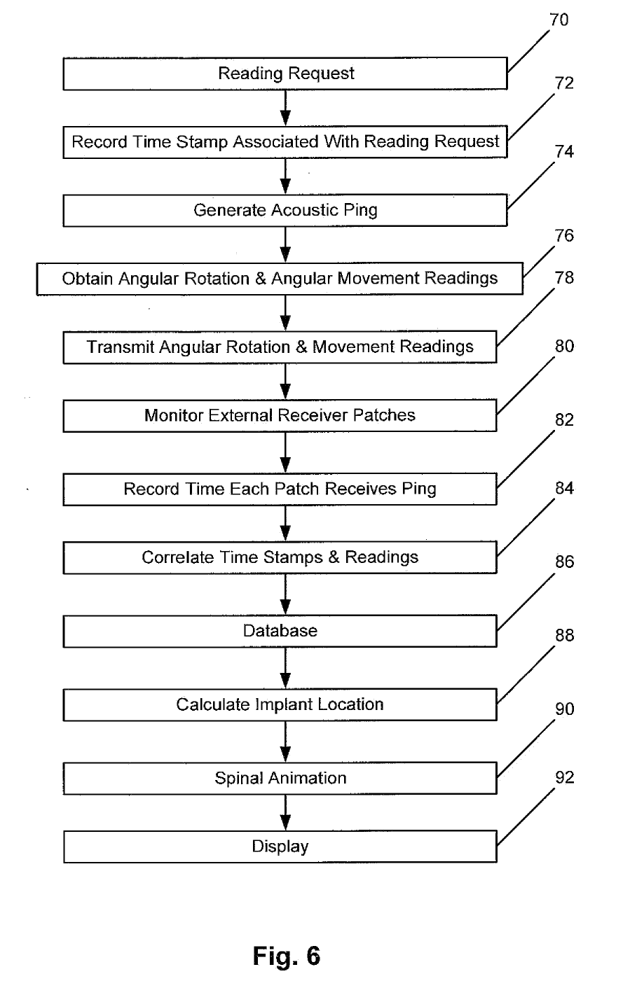

Referring to FIG. 6, during a representative procedure in which the motion of the vertebra 14 of the spine 12 is desired to be analyzed in connection with a patient, the external control unit 18 generates a plurality of reading requests that are transmitted to the signal emitting implant 16 being represented at step 70. The reading requests are transmitted to one or more implants 16 using the telemetry units 44, 64. At step 72, the external control unit 18 stores a time stamp associated with the reading request. In response to the reading request, the implants 16 generate an acoustic ping 24, which is represented at step 74. In addition, at step 76 the controller 40 of the implant 16 obtains an angular rotation reading and a angular movement reading from the gyroscope 46 and the accelerometer 48, respectively.

At step 78, the angular rotation and movement readings are transmitted from the implant 16 to the external control unit 18 using the respective telemetry units 44, 64. The external control unit 18 also monitors the external receiver patches 20 for detection of the acoustic pulse 24, which is illustrated at step 80. As each external receiver patch 20 detects the ping, the control unit 18 records the time associated with the receipt of each ping, which is represented at step 82. At step 84, the control unit 18 correlates the time date associated with the origination of the ping with the time dates associated with the receipt of each ping by the external receiver patches 20. In addition, the values read from the gyroscope 46 and accelerometer 48 are correlated with the time stamps. These values can then be stored in a database 86 associated with the analysis of the patient. The database 86 can be stored in the memory 68 of the control unit 18 in forms in which the control unit 18 comprises a portable desktop workstation. During the analysis, numerous samples can be taken to gather an in depth analysis of the movement of the spine 12.

An implant location application 88 can be included on the control unit 18 that is operable to determine the three-dimensional location of the implant 16 as a function of the time stamp associated with the ping and the time stamps associated with the receipt of a ping detection by the external receiver patches 20. The three-dimensional location of the implant 16 can be stored in the database 86. A spinal animation application 90 can be included on the control unit 18 that is configured to retrieve the values from the database 86 and generate a three-dimensional animation of the movement of the spine 12. In particular, the three-dimensional animation is generated as a function of the three-dimensional location of the implant 16, the gyroscope reading, and the accelerometer reading. Since a plurality of samples is taken, each sample provides a unique orientation of the vertebra 14 of the spine 12 thereby allowing the three-dimensional animation of the spine 12 in motion to be created. Once generated, the control unit 18 can include a display 92 upon which the three-dimensional animation of the spine 12 is generated so that a physician may review the spinal animation.

During implantation of the implants 16, the physician can designate where each respective implant 16 was installed on a setup screen generated by a setup application associated with the system 10. For example, if all of the implants 16 were placed on the lamina of the spine 12 the physician or assistant would designate such during setup. The exact vertebra 14 upon which the implants 16 are implanted may also be designated. In one form, replicas of the vertebra 14 are utilized by the animation application 90 to provide the physician with a realistic view of how the vertebra 14 is moving while the patient moves.

Although various embodiments have been described as having particular features and/or combinations of components, other embodiments are possible having a combination of any features and/or components from any of embodiments as discussed above. As used in this specification, the singular forms “a,” “an” and “the” include plural referents unless the context clearly dictates otherwise. Thus, for example, the term “a member” is intended to mean a single member or a combination of members, “a material” is intended to mean one or more materials, or a combination thereof.

While the invention has been illustrated and described in detail in the drawings and foregoing description, the same is to be considered as illustrative and not restrictive in character, it being understood that all changes and modifications that come within the spirit of the invention are desired to be protected.

What is claimed is:

1. A system, comprising:
a plurality of implants placed in a predetermined location on a plurality of respective vertebra of a patient, said implants each comprising:
a controller having a memory;
a telemetry unit connected with said controller; and
an acoustic generator configured to generate an acoustic pulse in response to a signal from said controller;
an external control unit in communication with said telemetry unit, wherein said external control unit is operable to generate a reading signal that is sent to said controller via said telemetry unit that causes said controller to generate said signal; and
at least three external receiving patches attached to an exterior surface of said patient and connected with said external control unit, wherein said external control unit is operable to generate a first time stamp associated with said reading signal and a plurality of unique time stamps associated with times at which said at least three external receiving patches detect said acoustic pulse.

2. The system of claim 1, wherein said external control unit is operable to generate a three-dimensional location reading as a function of said first time stamp and said plurality of unique time stamps.

3. The system of claim 1, wherein said first time stamp is associated with a time in which said signal is generated.

4. The system of claim 1, further comprising a gyroscope sensor connected with said controller configured to generate an angular rotation reading associated with each said implant.

5. The system of claim 4, wherein said angular rotation reading is transmitted to said control unit using said telemetry unit.

6. The system of claim 1, further comprising an accelerometer connected with said controller configured to generate an angular motion reading associated with each said implant.

7. The system of claim 6, wherein said angular motion reading is transmitted to said control unit using said telemetry unit.

8. The system of claim 7, wherein said angular motion reading corresponds to lateral bending and flexion extension of each said vertebra.

8. The system of claim 1, further comprising a spinal animation application configure to generate a spinal animation as a function of a plurality of readings taken from said implants.

9. A system, comprising:
at least one implant configured to be implanted on a vertebra of a spine, wherein said implant includes a controller connected with an acoustic generator that is operable to generate an acoustic pulse in response to a signal from said controller, wherein said implant includes a gyroscope connected with said controller configured to generate an angular rotation reading associated with said vertebra, wherein said implant includes an accelerometer connected with said controller configured to generate an angular motion reading associated with said vertebra, and a first telemetry unit connected with said controller;
an external control unit connected with a second telemetry unit in communication with said first telemetry unit of said implant, wherein said external control unit is connected with at least three external receiver patches placed on a patient's skin in a spaced apart relationship in relation to said at least one implant, wherein said external control unit is operable to generate a reading signal that is transmitted to said controller of said implant and associate a first time value with said reading signal; and
wherein in response to said reading signal said controller is operable to generate said signal to cause said acoustic generator to generate said acoustic pulse, wherein in response to said reading signal said controller obtains and transmits said angular rotation reading and said angular motion reading to said external control unit, wherein each of said external receiving patches are operable to generate detection signals that are transmitted to said external control unit when said acoustic pulse is detected by said external receiving patches, and wherein said external control unit is operable to assign a detection time value to each detection signal received from each respective external receiving patch.

10. The system of claim 9, wherein said external control unit includes a universal interface port for transmitting data to a computing device.

11. The system of claim 9, further comprising an implant location application operable to determine a three-dimensional location of said implant as a function of said first time value and said detection time values.

12. The system of claim 9, wherein a plurality of reading signals are generated to generate a plurality of result sets that are stored in a database.

13. The system of claim 12, wherein each said result set includes said first time value, said detection time values, said angular rotation reading and said angular motion reading.

14. The system of claim 13, further comprising an animation application configured to generate a spinal animation as a function of said plurality of result sets.

15. The system of claim 9, further comprising a temperature sensor connected with said controller for generating a temperature reading of tissue surrounding said at least one implant.

16. The system of claim 9, further comprising a rechargeable power unit connected with said controller, said gyroscope sensor, said accelerometer, and said acoustic generator.

17. The system of claim 16, further comprising an external power patch placed on said patient's skin for recharging said rechargeable power unit.

18. The system of claim 17, wherein said power patch is operable to generate electromagnetic energy that is used to charge said rechargeable power unit.

19. The system of claim 17, wherein said power patch is operable to generate acoustic waves that is used to charge said rechargeable power unit.

20. A method, comprising:
generating a reading request with an external control unit that is wirelessly transmitted to at least one implant oriented in a predetermined location on a vertebra of a spine;
recording a first time value associated with said reading request;
generating an acoustic ping with said implant in response to said reading request;
obtaining an angular rotation reading and a angular movement reading in response to said reading request;
wirelessly transmitting said angular rotation reading and said angular movement reading to said external control unit;
monitoring at least three external receiving patches connected with said external control unit for detection of said acoustic ping;
recording a detection time value from each said external receiving patch when said acoustic ping is detected;
storing said first time value, said angular rotation reading, said angular movement reading, and said detection time values in a database associated with said external control unit;
calculating a location value for said at least one implant as a function of said first time value and said detection time values; and
generating a graphical animation of said implants on said vertebra of said spine as a function of said location value, said angular rotation reading, and said angular movement reading.
